(19) United States
(12) Patent Application Publication
(10) Pub. No.: US 2023/0029542 A1
(43) Pub. Date: Feb. 2, 2023

(54) MEDICAL INFORMATION PROCESSING METHOD, MEDICAL INFORMATION PROCESSING DEVICE, AND PROGRAM

(71) Applicant: TERUMO KABUSHIKI KAISHA, Tokyo (JP)

(72) Inventors: Yoshihito MACHIDA, Sagamihara (JP); Nobuyuki TANIGAKI, Sagamihara (JP)

(73) Assignee: TERUMO KABUSHIKI KAISHA, Tokyo (JP)

(21) Appl. No.: 17/873,709

(22) Filed: Jul. 26, 2022

(30) Foreign Application Priority Data
Jul. 30, 2021 (JP) 2021-126158

(51) Int. Cl. G16H 10/60

(52) U.S. Cl. CPC G16H 10/60

(57) ABSTRACT

A medical information processing method that is executed by a processor, and includes: acquiring event information registered in a medical information system and including a patient identifier (ID), the patient ID including identification information on a patient, identification information on an event, and first temporal information indicating occurrence time of the event; acquiring, by using the patient ID and the first temporal information, first medical care information including second temporal information with a temporal resolution higher than that of the first temporal information related to the patient; and estimating, based on the first medical care information, the occurrence time of the event with the temporal resolution higher than that of the first temporal information.

### CROSS-REFERENCES TO RELATED APPLICATIONS

This application claims priority to Japanese Application No. 2021-126158 filed on Jul. 30, 2021, the entire content of which is incorporated herein by reference.

### TECHNOLOGICAL FIELD

This disclosure relates to a medical information processing method, a medical information processing device, and a program.

### BACKGROUND DISCUSSION

In recent years, the application of artificial intelligence to the medical field has progressed. For example, a technique has been proposed of generating a learning data set that includes a plurality of sets of data on a medical record and medical care information including vital data on a patient before the creation of the medical record, and performing machine learning using the learning data set, thereby generating a learned model (for example, see Japanese Patent Application Publication No. 2021-86558 A). Accordingly, an occurrence of an event to be described in the medical record can be predicted using the medical care information such as the vital data as an input. Such a device can be used for predicting a prognosis of the patient, supporting a medical doctor in forming treatment planning, and the like.

In a case where machine learning is performed using retrospective medical data accumulated in a medical information system of a medical institution, information on a medical record such as an electronic medical record only includes date information on a prediction target such as an occurrence of an event but does not include information on the precise time in many cases. For example, in a case where data such as a dialysis shift and introduction/weaning of a ventilator has been recorded on an electronic medical record as medical-receipt data for a request of medical service fees, information on the date is included but information on the time is not included in many cases. Moreover, a record of death includes information on the date of the death but does not include the time information in many cases.

Meanwhile, a state of a patient changes suddenly, for example, in an intensive care unit (ICU) where serious patients are treated in some cases. Therefore, in order to predict and manage a change in the state of the patient, the prediction based on a temporal resolution higher than the unit of day is needed. However, even if an occurrence of an event is predicted using a learned model generated by setting event information in the unit of day accumulated in the electronic medical record as objective variables, only the prediction with a low temporal resolution can be made, so that there is a concern that the prediction is insufficient for the use in the medical care of the patient.

### SUMMARY

Accordingly, this disclosure focuses on these circumstances to generate data for machine learning for predicting an event occurrence of a patient with a temporal resolution higher than that of temporal information included in the information registered in a medical information system such as an electronic medical record.

A medical information processing method as one aspect in this disclosure is a medical information processing method that is executed by a computer, and: acquires event information registered in a medical information system and including a patient identifier (ID) that is identification information on a patient, identification information on an event, and first temporal information indicating occurrence time of the event; acquires, by using the patient ID and the first temporal information, first medical care information including second temporal information with a temporal resolution higher than that of the first temporal information related to the patient; and estimates, based on the first medical care information, the occurrence time of the event with the temporal resolution higher than that of the first temporal information.

As one embodiment, the first medical care information includes vital data on the patient.

As one embodiment, the medical information processing method includes estimating the occurrence time of the event, based on the second temporal information when a value of data of a predetermined data item in the vital data has achieved a predetermined change.

As one embodiment, the medical information processing method includes estimating the occurrence time of the event, based on the second temporal information when the acquired data item in the vital data has been changed.

As one embodiment, the first medical care information includes drug administration data indicating information on administration of a drug performed relative to the patient.

As one embodiment, the first medical care information includes test value data indicating a result of a test performed relative to the patient.

As one embodiment, the first medical care information includes time-series data that is generated on a time-series basis by the second temporal information, and the medical information processing method includes estimating the occurrence time of the event based on a change in a time interval in which the time-series data is generated.

As one embodiment, the first medical care information includes text data input to an electronic medical record or another medical system, and the second temporal information when the text data has been input.

As one embodiment, the medical information processing method includes estimating the occurrence time of the event based on a combination of two or more types of the first medical care information.

As one embodiment, the event includes a specific event that is mapped with one or more specific data items included in the first medical care information, and the medical information processing method includes estimating the occurrence time of the specific event based on only the specific data item.

As one embodiment, the medical information processing method includes estimating the occurrence time of the event using a first learned model in which the first medical care information is set as an input and the occurrence time of the event is set as an output.

As one embodiment, the medical information processing method includes setting the event as a first event, setting information including the first event and the estimated occurrence time as first event information, using the first event information as the first medical care information, and estimating occurrence time of a second event different from the first event.

As one embodiment, the medical information processing method includes acquiring second medical care information that is used as input data of a machine learning model that predicts occurrence of the event, and mapping the second medical care information with the estimated occurrence time of the event.

As one embodiment, the medical information processing method includes mapping the second medical care information with the occurrence time of the event by adding information indicating the occurrence time of the event to the second medical care information.

As one embodiment, the medical information processing method includes setting the event information on a plurality of patients and the second medical care information mapped with the occurrence time of the event as teacher data, and generating a second learned model in which the second medical care information is set as an input and the event information is set as an output using the teacher data.

As one embodiment, the medical information processing method includes acquiring third medical care information on a specific patient, inputting the third medical care information to the second learned model, and predicting occurrence of an event related to the specific patient.

As one embodiment, the medical information system is an electronic medical record system.

A medical information processing device as one aspect in this disclosure includes a processor: acquires event information registered in a medical information system and including a patient ID that is identification information on a patient, identification information on an event, and first temporal information indicating occurrence time of the event; acquires, by using the patient ID and the first temporal information, first medical care information including second temporal information with a temporal resolution higher than that of the first temporal information related to the patient; and estimates, based on the first medical care information, the occurrence time of the event with the temporal resolution higher than that of the first temporal information.

A non-transitory computer readable program as one aspect in this disclosure causes a computer to execute processing of: acquiring event information registered in a medical information system and including a patient ID that is identification information on a patient, identification information on an event, and first temporal information indicating occurrence time of the event; acquiring, by using the patient ID and the first temporal information, first medical care information including second temporal information with a temporal resolution higher than that of the first temporal information related to the patient; and estimating, based on the first medical care information, the occurrence time of the event with the temporal resolution higher than that of the first temporal information.

In the medical information processing method, the medical information processing device, and the program in this disclosure, occurrence time of an event is estimated based on first medical care information including second temporal information with a temporal resolution higher than first temporal information registered in a medical information system. Accordingly, with this disclosure, it is possible to generate data for machine learning for predicting an event occurrence of a patient with a temporal resolution higher than temporal information included in information registered in the medical information system.

### BRIEF DESCRIPTION OF THE DRAWINGS

FIG. 1 is a diagram illustrating a schematic configuration of an inter-medical institution system including a medical information processing device according to one embodiment.

FIG. 2 is a block diagram illustrating one example of a schematic configuration of the medical information processing device in FIG. 1.

FIG. 3 is a diagram illustrating an overall process of machine learning, in the inter-medical institution system in FIG. 1.

FIG. 4 is a flowchart of processing that is executed by a control unit of the medical information processing device in FIG. 1.

FIG. 5 is a flowchart illustrating estimation processing of event occurrence time in FIG. 4.

FIG. 6 is a diagram illustrating one example of an extraction order of first medical care information.

FIG. 7 is a diagram for explaining one example of a method of estimating introduction time of artificial ventilation.

FIG. 8 is a diagram illustrating one example of mapping of the first medical care information with event occurrence time.

FIG. 9 is a diagram illustrating one example of a mapping relation of the event and the first medical care information.

FIG. 10 is a diagram for explaining one example of a method of estimating time of death.

FIG. 11 is a schematic configuration diagram illustrating one example of an embodiment in which devices are dispersedly arranged inside and outside of a medical institution.

### DETAILED DESCRIPTION

Set forth below with reference to the accompanying drawings is a detailed description of embodiments of a medical information processing method, a medical information processing device, and a program. Note that since embodiments described below are preferred specific examples of the present disclosure, although various technically preferable limitations are given, the scope of the present disclosure is not limited to the embodiments unless otherwise specified in the following descriptions.

### Configuration of Inter-Medical Institution System

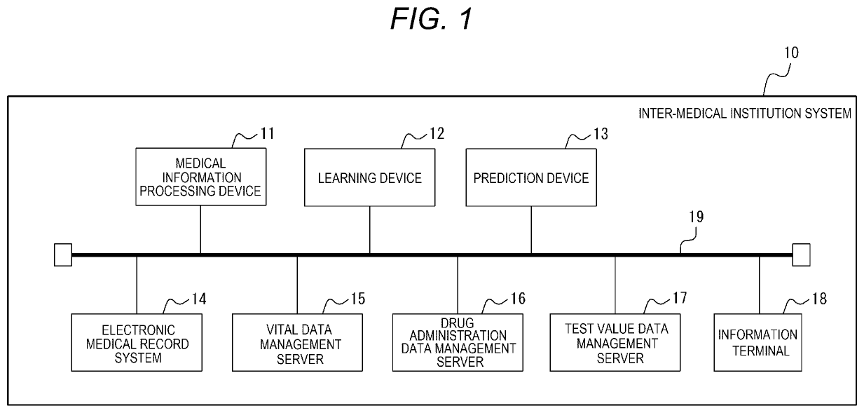

A medical information processing device 11 according to one embodiment of this disclosure can be included in an inter-medical institution system 10, as illustrated in FIG. 1. The inter-medical institution system 10 includes a system that predicts, by performing machine learning using event information to be registered in a medical information system in a medical institution as an output (objective variable) and medical care information as an input (explanatory variable), an event to occur in a patient from the medical care information. The medical information system can include all the devices and systems that deal with information on patients. The medical information system can include, for example, in the medical institution and the like, a medical-receipt creation computer, an electronic medical record system, and a system such as an ordering system that supports medical office work and medical care, and a computer that keeps information on patients in some form. Hereinafter, the medical information system will be described by being regarded as an electronic medical record system.

In the present application, an event indicates a relatively large state change generated in a patient. The event is information with a diagnosis by a medical doctor, and/or information on a procedure by the medical doctor. The event to be registered in an electronic medical record can include the occurrence of complications of the patient, death, the occurrence of blood poisoning and other infectious diseases, shock, the execution of a procedure to the patient, and the like. The complications can include, for example, cerebral infarction, intracranial bleeding, myocardial infarction, diabetes insipidus, hyponatremia, hypernatremia, renal dysfunction, liver dysfunction, and the like. The procedure to the patient can include, for example, the introduction of a dialysis and the introduction and weaning of a ventilator, and the like.

The medical care information is data relating to medical care for a patient. The medical care information can include, for example, data of an electronic medical record, vital data on a patient, drug administration data, test value data, and other data relating to the medical care for the patient. The vital data can include, for example, measurement items of blood pressure, respiratory rate, heart rate, body temperature, arterial oxygen saturation (SpO₂), urine volume, and the like. The vital data can include, for example, information on the time when the vital data was measured, as time information. The drug administration data can include, for example, identification information on a drug administered to the patient, and information on the dose of the drug administered to the patient. The drug administration data can include, for example, information on the time when the drug was administered, as time information. The test value data can include, for example, data indicating various sorts of test results acquired from various sorts of tests including, for example, a blood test, an electrocardiogram test, a chest X-RAY test, and the like. The test value data can include, for example, information on the time when the test was performed, as time information. Hereinafter, individual measurement items of the vital data, respective drugs in the drug administration data, and respective test items in the test value data, which are included in the medical care information, are called data items. The data items in the medical care information can be put in another way as the variables in the medical care information.

The inter-medical institution system 10 can include, for example, in addition to the medical information processing device 11, a learning device 12, a prediction device 13, an electronic medical record system 14, a vital data management server 15, a drug administration data management server 16, a test value data management server 17, and an information terminal 18. The respective devices can be computers that are mutually connected so as to be communicable via a network 19. As for the network 19, for example, a wired local area network (LAN) and a wireless LAN can be used. The computers can include a personal computer (PC), a PC server, a work station, a general-purpose computer, and other computers.

The medical information processing device 11 is configured to be capable of collecting event information from the electronic medical record system 14. The event information can include, for example, a patient ID that is identification information on a patient, identification information on an event, and date information on occurrence date of the event. The date information is first temporal information in this disclosure. Moreover, the medical information processing device 11 is configured to be capable of acquiring, from the vital data management server 15, the drug administration data management server 16, and the test value data management server 17, first medical care information on an event occurrence date of the patient corresponding to the event information, with the time information. The time information is second temporal information in this disclosure. The first medical care information is medical care information to be used for estimating occurrence time of the event. The occurrence time of the event is information on occurrence time of the event, and has a temporal resolution higher than that of the date information. The medical information processing device 11 may search time information included in the medical care information based on the date information on the event occurrence date to acquire first medical care information including information on the occurrence date of the event. The medical information processing device 11 can estimate occurrence time of the event with a temporal resolution higher than the unit of day, based on the acquired first medical care information. The medical information processing device 11 maps the second medical care information with the estimated occurrence time of the event. The second medical care information is medical care information to be used in input data of the machine learning. The medical information processing device 11 may be operated, for example, by an operator who is in charge of collection, processing, and the like of data.

Each of the first medical care information and the second medical care information includes data of one or more data items. The first medical care information and the second medical care information may be the same information, or may be information in which parts of the first medical care information and the second medical care information are overlapped with and the other parts of the first medical care information and the second medical care information are different from each other. Moreover, the first medical care information and the second medical care information may be entirely different from each other. As the first medical care information, in order to specify occurrence time of a specific event, data in the data item that is directly related to the event and changes between before and after the event is selected with priority. Meanwhile, as the second medical care information, data in the data item that is estimated to have causality relative to the occurrence of the event is selected with priority. For example, as the first medical care information, data in the data item that is included in the vital data including information on the occurrence date of the event may be selected, and as the second medical care information, data in the data item that is included in the drug administration data for the patient may be selected. The second medical care information may include data on the treatment and the procedure by the medical doctor to be described in the electronic medical record.

The learning device 12 sets a large number of pieces of event information on a large number of patients and the second medical care information mapped with the occurrence time of respective events as teacher data, and performs machine learning using the teacher data, thereby generating a learned model (second learned model). The event information is an objective variable of the machine learning, and the second medical care information is an explanatory variable of the machine learning. The learning device 12 provides the generated learned model to the prediction device 13.

The prediction device 13 uses the learned model generated by the learning device 12 to predict an event that occurs in the patient. In other words, the prediction device 13 predicts a prognosis of the patient. The prediction device 13 acquires third medical care information on a specific patient who is an object of prediction. The third medical care information can include, for example, medical care information of data items (variables) as same as those of the second medical care information. The prediction device 13 inputs the acquired third medical care information to the learned model generated by the learning device 12, and outputs information on an event including the event to be predicted and the occurrence time the event.

The functions of the medical information processing device 11, the learning device 12, and the prediction device 13 may be mounted on the same hardware, not on the separate hardware. For example, all the functions of the medical information processing device 11, the learning device 12, and the prediction device 13 may be implemented by a single computer. Moreover, for example, the functions of the medical information processing device 11 and the learning device 12 may be implemented by a single computer that executes a learning phase of the machine learning different from that of the prediction device 13.

The electronic medical record system 14 manages data on electronic medical records that are used in the medical institution. The electronic medical record system 14 is included in the medical information system. Information on an event in a patient can be input to the electronic medical record, for example, by a medical doctor or a nurse. The information on the event to be input to the electronic medical record system 14 can include date information on the date when the event has occurred, but does not necessarily include time information, which is more detailed than the date. A part of the medical care information on the patient may be input to the electronic medical record system 14. In the electronic medical record system 14, findings of the clinical examination input by the medical doctor as text, prescription of a drug, and the content of a procedure, and the like may be recorded with the time information.

Comments as text input by a medical doctor or a nurse may be included in another medical system other than the electronic medical record system, in some cases. For example, as another medical system, a system that manages an image of the computed tomography (CT), the magnetic resonance imaging (MRI), or the like is included. The comments input to these medical systems are registered with the time information filled out by the system, in some cases. The medical information processing device 11 may be configured to be capable of acquiring information from these other medical systems.

The vital data management server 15, the drug administration data management server 16, and the test value data management server 17 respectively manage vital data, drug administration data, and test value data on each patient. The vital data management server 15, the drug administration data management server 16, and the test value data management server 17 may be respectively configured as data bases on which the data base management systems are mounted.

The vital data, the drug administration data, and the test value data may be managed by a single server, not by the different servers as in FIG. 1. Moreover, the vital data, the drug administration data, and the test value data may be managed by a plurality of different hardware in various sorts of combinations. The medical care information may be classified and managed by a method different from that described herein. At least parts of the vital data, the drug administration data, and the test value data are stored and managed by the electronic medical record system 14.

The information terminal 18 can be a terminal that is used by a health care worker such as a medical doctor or a nurse in a consultation room and the like. As for the information terminal 18, a PC can be used, for example. The information terminal 18 can display, add, and change the content of an electronic medical record that is managed by the electronic medical record system 14. The information terminal 18 can display each data that is managed by the vital data management server 15, the drug administration data management server 16, and the test value data management server 17. The information terminal 18 can transmit an instruction of prediction about an event occurrence of a patient to the prediction device 13, and acquire a result predicted by the prediction device 13.

### Configuration of Medical Information Processing Device

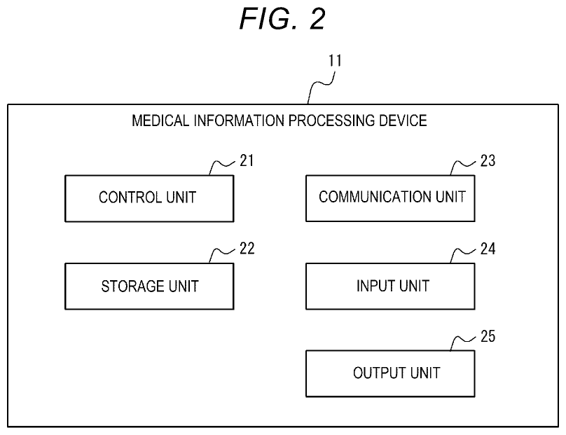

The medical information processing device 11 can be provided with, as illustrated in FIG. 2, a control unit 21, a storage unit 22, a communication unit 23, an input unit 24, and an output unit 25. Note that, similar to the medical information processing device 11, each of the learning device 12, the prediction device 13, the electronic medical record system 14, the vital data management server 15, the drug administration data management server 16, and the test value data management server 17 may be configured to include a control unit, a storage unit, a communication unit, an input unit, and an output unit.

The control unit 21 includes at least one processor. The processor includes a general-purpose processor such as a central processing unit (CPU), or a dedicated processor that is specialized in specific processing. The control unit 21 may include an application specific integrated circuit (ASIC), a digital signal processor (DSP), a programmable logic device (PLD), a field-programmable gate array (FPGA), or a combination of the application specific integrated circuit (ASIC), the digital signal processor (DSP), the programmable logic device (PLD), and the field-programmable gate array (FPGA). The control unit 21 may include a memory that is embedded in the processor or a memory that is independent of the processor. The control unit 21 executes processing relating to an operation of the medical information processing device 11 while controlling the respective units of the medical information processing device 11.

The storage unit 22 can include at least one semiconductor memory, at least one magnetic memory, at least one optical memory, or a combination of the at least one semiconductor memory, the at least one magnetic memory, and the at least one optical memory. The semiconductor memory can be, for example, a random access memory (RAM) or a read only memory (ROM). The RAM is, for example, a static random access memory (SRAM) or a dynamic random access memory (DRAM). The ROM can be, for example, an electrically erasable programmable read only memory (EEPROM). The storage unit 22 can function as, for example, a main storage device, an auxiliary storage device, or a cache memory. In the storage unit 22, data to be used for an operation of the medical information processing device 11, and data obtained by the operation of the medical information processing device 11 are stored.

The communication unit 23 includes at least one communication interface. The communication interface can be, for example, a LAN interface, an interface compatible with a mobile communication standard such as long term evolution (LTE), 4th generation (4G) standard, or 5th generation (5G) standard, or an interface compatible with the near field communication standard such as Bluetooth®. The communication unit 23 receives data to be used for the operation of the medical information processing device 11, and transmits data to be obtained by the operation of the medical information processing device 11. The medical information processing device 11 can acquire event information that is included in an electronic medical record (medical information system), and first medical care information including the vital data, the drug administration data, and the test value data, via the communication unit 23.

The input unit 24 can include at least one input interface. The input interface can be, for example, a physical key (i.e., keyboard), an electrostatic capacitance key, a pointing device, a touch screen integrally provided to a display, an imaging device such as a camera, or a microphone. The input unit 24 receives an operation of inputting data that is used for the operation of the medical information processing device 11. The input unit 24 receives an operation by an operator to the event information and the first medical care information. The input unit 24 may be connected to the medical information processing device 11 as an external input device, instead of being provided to the medical information processing device 11.

The output unit 25 includes at least one output interface. The output interface can be, for example, a display or a speaker. The display can be, for example, a liquid crystal display (LCD) or an organic electro luminescence (EL) display. The output unit 25 outputs data to be obtained by the operation of the medical information processing device 11. The output unit 25 may be connected to the medical information processing device 11 as an external output device, instead of being provided to the medical information processing device 11.

The function of the medical information processing device 11 can be implemented such that a processor serving as the control unit 21 executes a program according to the present embodiment. In other words, the function of the medical information processing device 11 can be, for example, implemented by software. The program causes the computer to execute the operation of the medical information processing device 11, and thus causes the computer to function as the medical information processing device 11. In other words, the computer functions as the medical information processing device 11 by executing the operation of the medical information processing device 11 in accordance with the program.

The program can be stored in a non-temporary computer-readable medium. The non-temporary computer-readable medium can be, for example, a flash memory, a magnetic recording device, an optical disk, a magneto-optical medium, or a ROM. The program can be distributed, for example, by selling, transferring, or lending a transportable medium in which the program is stored, such as a secure digital (SD) card, a digital versatile disc (DVD), or a compact disc read only memory (CD-ROM). The program may be distributed by storing the program in a storage of a server, and transferring the program from the server to another computer. The program may be provided as a program product.

The computer temporarily stores, for example, a program stored in the transportable medium or a program transferred from the server, in the main storage device. Further, the computer reads the program stored in the main storage device by the processor, and executes processing in accordance with the read program by the processor. The computer may directly read a program from the transportable medium, and may execute processing in accordance with the program. Every time when the program is transferred to the computer from the server, the computer may successively execute processing in accordance with the received program. The processing may be executed by so-called application service provider (ASP) type service in which the program is not transferred from the server to the computer, but the function is implemented only by an execution instruction and a result acquisition. The program includes information that is used for processing by an electronic calculator and conforms to the program. For example, data that is not a direct command to the computer but has a property to define the processing by the computer corresponds to “information that conforms to the program”.

A part or all of the functions of the medical information processing device 11 may be implemented by a programmable circuit or a dedicated circuit serving as the control unit 21. In other words, a part or all of the functions of the medical information processing device 11 may be implemented by hardware.

### Overall Process of Machine Learning

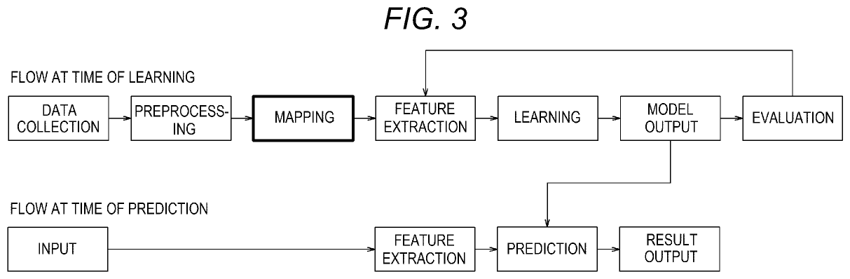

With reference to FIG. 3, a process of the machine learning to be executed in the inter-medical institution system 10 will be described. The process of the machine learning can include a flow at the time of learning and a flow at the time of prediction.

The flow at the time of learning can include respective processes of “data collection”, “preprocessing”, “mapping”, “feature extraction”, “learning”, “model output”, and “evaluation”.

The “data collection” includes a process of collecting event information from the electronic medical record system 14, and a process of collecting medical care information from the vital data management server 15, the drug administration data management server 16, and the test value data management server 17. The medical care information is used for first medical care information and/or second medical care information. The operator can collect these pieces of information using the medical information processing device 11.

The “preprocessing” is a process of organizing the collected event information and medical care information to make it easier to perform machine learning. The “preprocessing” includes cleansing of data, integration of data, and conversion of data. These processes in the preprocessing are known in the field of machine learning, and thus explanations of the cleansing of data, the integration of data, and the conversion of data are omitted. The medical information processing device 11 may include software that supports the preprocessing to be executed by the operator.

The “mapping” is a process of estimating occurrence time of an event with a temporal resolution higher than the unit of day based on the medical care information, and mapping the medical care information with the estimated occurrence time of the event. The “mapping” is processing to be a characteristic of the medical information processing method that is performed by the medical information processing device 11 in this disclosure. The estimation of the occurrence time of the event is executed based on the first medical care information extracted from the entire medical care information. The mapping of the medical care information with the occurrence time of the event can be performed, for example, by providing a flag region indicating the occurrence of the event in the medical care information collected from each device, and setting the flag region at the event occurrence time to a predetermined value. Moreover, the “mapping” may be performed by adding information on the time to the event information. The “mapping” can be put in another way as labeling.

The “feature extraction” is a process of extracting second medical care information necessary for machine learning among the data of the medical care information, and determining the second medical care information serving as an input of the machine learning. The extraction of the second medical care information can be performed by extracting a data item in the medical care information that is estimated to have an influence on the occurrence of the event. The operator can execute the “feature extraction” using the learning device 12. The learning device 12 may automatically execute the feature extraction based on the mapped event information and medical care information, independent of the operator.

The “learning” is a process of executing machine learning using teacher data in which the event information is used as an objective variable and the second medical care information extracted from the entire medical care information is used as an explanatory variable, and generating a learned model. As the machine learning, for example, a method such as Recurrent Neural Network (RNN), Random Forest, Gradient Boosting, or Support Vector Machine (SVM) can be used. The “learning” is executed by the learning device 12. The learning device 12 may simultaneously execute the “feature extraction” and the “learning”.

The “model output” is a process of outputting a learned model for the flow at the time of prediction. The learning device 12 may transmit a learned model to the prediction device 13 via the network 19.

The “evaluation” is a process of evaluating a learned model. The learning device 12 evaluates the prediction accuracy of a learned model using part of the data extracted in the feature extraction as test data. In a case where the prediction accuracy is relatively low, the learning device 12 may cause the processing to return to the “feature extraction” and may change a data item to be used for the machine learning. In a case where the prediction accuracy is relatively low, the learning device 12 may cause the processing to return to the process of the “preprocessing” or the “mapping”.

The flow at the time of prediction includes respective processes of “input”, “feature extraction”, “prediction”, and “result output”. The processing at the time of prediction may be started, for example, by a medical doctor, a nurse, or a specialized operator transmitting an instruction from a terminal device to the prediction device 13.

The “input” is a process of acquiring medical care information on a patient who is to be predicted. When the prediction device 13 receives an instruction to predict an occurrence of an event about a patient from the information terminal 18, the prediction device 13, for example, acquires medical care information on the patient from the electronic medical record system 14, the vital data management server 15, the drug administration data management server 16, the test value data management server 17, and the like.

The “feature extraction” is a process of extracting data on third medical care information to be used for the prediction from the medical care information on the patient. The prediction device 13 extracts data in the data item the same as that of the second medical care information extracted in the “feature extraction” in the learning phase, as input data from the input medical care information to the learned model. Alternatively, as part of the third medical care information, in order to predict an effect by the procedure, the drug administration, and the like, information on the procedure, the drug administration, and the like that have not been executed yet may be input from the information terminal 18.

The “prediction” is a process of inputting the third medical care information as input data to the learned model generated in the learning phase, and performing prediction about an event occurrence. The prediction device 13 executes the “prediction”.

In the “result output”, the prediction device 13 outputs a prediction result. The prediction device 13 outputs an event to be predicted, and occurrence time of the event to be predicted. The prediction device 13 may output the prediction result to a display device of the information terminal 18 that is operated, for example, by the medical doctor. The medical doctor can examine a medical care plan based on the prediction result.

### Processing Flow of Medical Information Processing Device

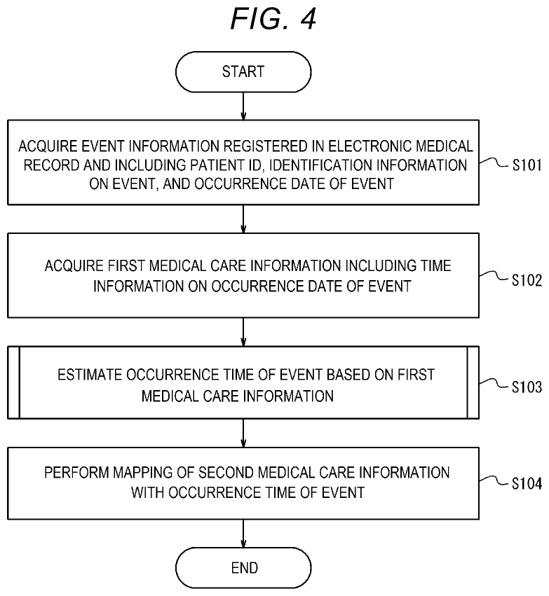

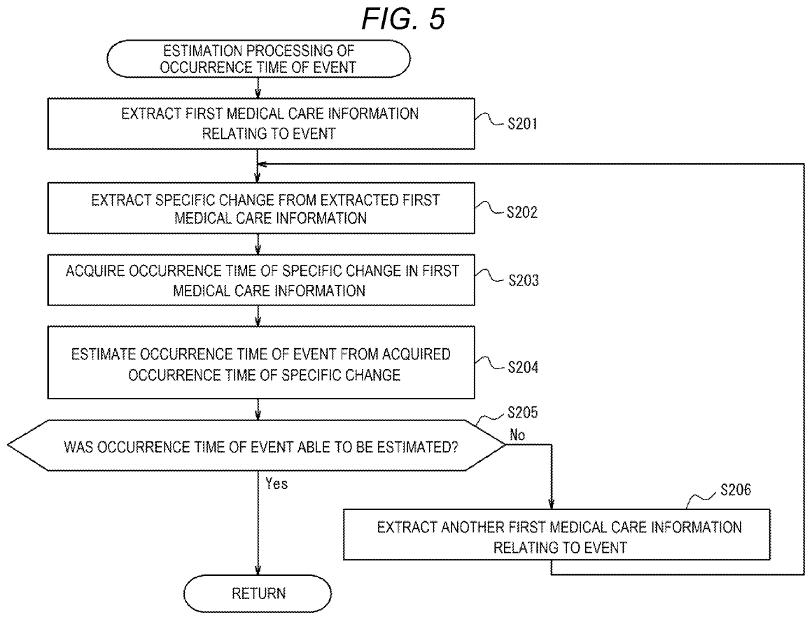

Hereinafter, with reference to FIGS. 4 and 5, among the processing that is executed by the medical information processing device 11, a processing content relating to the “mapping” that is a characteristic in this disclosure will be described. An operator may execute the processing of the flowcharts in FIGS. 4 and 5 using the input unit 24 and the output unit 25 of the medical information processing device 11, while operating the medical information processing device 11. Moreover, the processing of the flowcharts in FIGS. 4 and 5, for example, may be partially automated or may be totally automated.

Firstly, the control unit 21 of the medical information processing device 11 acquires, from the electronic medical record system 14, event information registered in an electronic medical record (medical information system) of a patient and including a patient ID that is identification information on the patient, identification information on an event, and date information on occurrence date of the event (Step S101). The control unit 21 may store the acquired event information in the storage unit 22. This processing may be executed at the process of the “data collection” in FIG. 3.

Next, the control unit 21 acquires first medical care information relating to the abovementioned event using the patient ID and the date information (Step S102). The control unit 21 can search medical care information using the patient ID and the date information as search conditions. The control unit 21 may acquire, from the entire medical care information, medical care information in which date information included in the time information matches date information included in the event information on the patient. For example, the first medical care information to be acquired can include time-series vital data, drug administration data, or test value data on the day when the event has occurred in the patient. The control unit 21 may store the acquired first medical care information in the storage unit 22. The medical information processing device 11 may acquire first medical care information by searching the medical care information acquired in the process of “data collection” in FIG. 3. Alternatively, the medical information processing device 11 may acquire first medical care information, for example, by directly searching the vital data management server 15, the drug administration data management server 16, and the test value data management server 17.

Next, the control unit 21 estimates occurrence time of the event based on the first medical care information, for each pair of the event information and the first medical care information (Step S103). In other words, although the event information only includes information on the date when the event has occurred in many cases, the control unit 21 estimates the time when the event has occurred with a temporal resolution higher than the date by using the first medical care information.

An estimation method of occurrence time of an event in the medical information processing device 11 will be described with reference to a flowchart of FIG. 5.

Firstly, the control unit 21 extracts first medical care information that is relating to the event or is estimated to have a high relationship with the event, based on the content of the event (Step S201). For example, the control unit 21 acquires time-series vital data on the date when the event has occurred. It can be considered that the vital data is information with the highest priority that can be used when the occurrence time of the event is estimated.

Next, the control unit 21 extracts a specific change from the extracted first medical care information (Step S202). The specific change can include a change in the value of data in the specific data item corresponding to the event, a change in the acquisition frequency of data, and a change in the data item.

The change in the value of data in the specific data item can include cases where the value of data in the specific data item that is included in the medical care information, for example, becomes an abnormal value, suddenly increases, suddenly decreases, and other cases. For example, when the medical care information is vital data, the change in the value of data in the specific data item includes cases where the blood pressure, the heart rate, the body temperature, or the like of the patient exceeds a range of the normal value, and becomes lower than the range of the normal value. When the first medical care information is drug administration data, the change in the value of data in the specific data item can include, for example, a case where the dose of a specific drug has increased.

The change in the acquisition frequency of data can include a change in the interval of time when the data has been acquired. For example, the change in the acquisition frequency of data can include a change in the measurement frequency of vital data. For example, for a patient whose condition is stable, vital data is measured for every two to three hours in some cases. In contrast, in a case where the condition of a patient has become worse, vital data is measured at a shorter interval in some cases. Accordingly, it is possible to estimate that an event has occurred because the time interval of the measurement of vital data has become shorter.

The change in the data item can include, for example, a case where data in the data item not acquired before is newly acquired. For example, end-tidal carbon dioxide tension (EtCO₂) is an index for evaluating whether the patient can perform ventilation. An EtCO₂ monitor that measures EtCO₂ can be used for management of a respiratory status of a patient under the mechanical ventilator management. Accordingly, it is estimated that the timing when EtCO₂ has been acquired as a data item of the vital data as the timing when a mechanical ventilator has been introduced.

If the control unit 21 has extracted a specific change from the first medical care information at Step S202, the control unit 21 acquires information on the time when the specific change has occurred (Step S203).

The control unit 21 estimates time when the event has occurred from the acquired information on the occurrence time of the specific change (Step S204). In several events, the occurrence time of the specific change acquired at Step S203 is estimated to be identical with the time when the event has occurred. Moreover, in other several events, the occurrence time of the event is estimated by considering the presence of a time lag from the occurrence time of the specific change acquired at Step S203 to the time when the event has occurred.

If the occurrence time of the event was able to be estimated at Step S204 (Step S205: Yes), the control unit 21 causes the processing to return to the flowchart in FIG. 4, and continues the processing. If the occurrence time of the event was unable to be estimated at Step S204 (Step S205: No), the control unit 21 extracts another first medical care information that is estimated to be relating to the event with a relatively high possibility (Step S206), and repeats the processing at Step S202 and the subsequent processing.

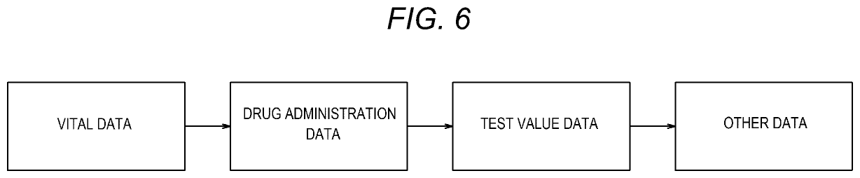

For example, as illustrated in FIG. 6, the control unit 21 may firstly acquire vital data. If the occurrence time of the event is unable to be estimated only from the vital data, the control unit 21 may successively acquire drug administration data, test value data, and other data as other first medical care information, and may execute the processing from Steps S202 to S205. However, the order for acquiring first medical care information is not limited to this order. The storage unit 22 may store the acquisition order of the first medical care information optimized in accordance with the event, and the control unit 21 may acquire the first medical care information based on this order.

Other data in FIG. 6 may include, for example, text data that indicates the findings, the prescription, the procedure, and the like by the medical doctor and is included in the electronic medical record or another medical information system, and information on the input time of the text data. The control unit 21 may acquire information having a relatively high relationship with the event from the text information included in the electronic medical record using the text mining technique, and may estimate the occurrence time of the event from the input time of the text.

The control unit 21 may estimate the occurrence time of the event based on a combination of two or more types of data among the data included in the vital data, the drug administration data, the test value data, and other data.

After the control unit 21 has estimated the occurrence time of the event at Step S205 in FIG. 5, as illustrated in FIG. 4, the control unit 21 performs mapping of the second medical care information with the occurrence time of the event (Step S104). For example, the control unit 21 can map the second medical care information with the occurrence time of the event by assigning a flag indicating the occurrence of the event to data at the occurrence time of the event among the time-series second medical care information. Alternatively, the control unit 21 can assign time information to the event information itself, and can indirectly map the second medical care information with the occurrence time of the event. Data in a plurality pieces of second medical care information that is used as an input (explanatory variable) in the process of learning at the post stage may be mapped with the event information (objective variable).

### Specific Example of Estimation and Mapping of Event Occurrence Time

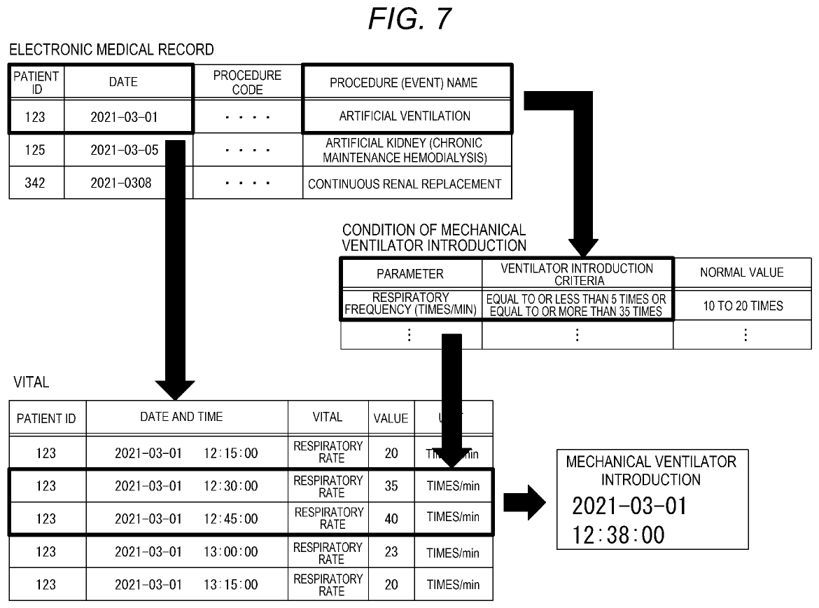

With reference to FIG. 7, one example of the estimation method of event occurrence time and mapping of the event occurrence time with second medical care information will be described. The control unit 21 of the medical information processing device 11 acquires, from information on an electronic medical record (medical information system), identification information on an event that has occurred in a patient, a patient ID that is identification information on the patient, and date information on occurrence date of the event. As for identification information on the event, a name of the event can be used. In a case of an example in FIG. 7, the control unit 21 acquires event information indicating that the artificial ventilation was introduced into a patient having a patient ID 123 on Mar. 1, 2021.

The control unit 21 can refer to various sorts of references in order to estimate the time when the event has occurred. Such the references may be stored in advance in the storage unit 22. For example, in a case of introduction of a mechanical ventilator, the respiratory rate is medical care information relating to the introduction of the mechanical ventilator. The ventilator introduction criteria indicating that a mechanical ventilator should be introduced when the respiratory frequency, for example, becomes 5 times or less or 35 times or more per one minute has been known. Accordingly, when the respiratory frequency becomes 35 times or more per one minute, it may be estimated that the medical doctor introduces a mechanical ventilator. As a reference, the reference determined by the nation and the public institution, the reference determined in the medical institution, the reference derived from the past medical care information in the medical institution, and the like can be used.

The control unit 21 searches vital data as first medical care information using a patient ID and date information in the electronic medical record, and acquires the vital data including data on an event occurrence date of the patient. The control unit 21 refers to the value of the respiratory rate included in the vital data, and extracts data in which the respiratory frequency changes from less than 35 times to 35 times or more per one minute. In the illustrated example, the respiratory frequency of the patient at 12:30 indicates 35 times.

The control unit 21 estimates introduction time of the mechanical ventilator by considering the time when the respiratory frequency in the vital data has exceeded the reference, a time necessary for introducing the ventilator, and other medical care information. In the illustrated example, the introduction time of the mechanical ventilator is estimated to 12:38.

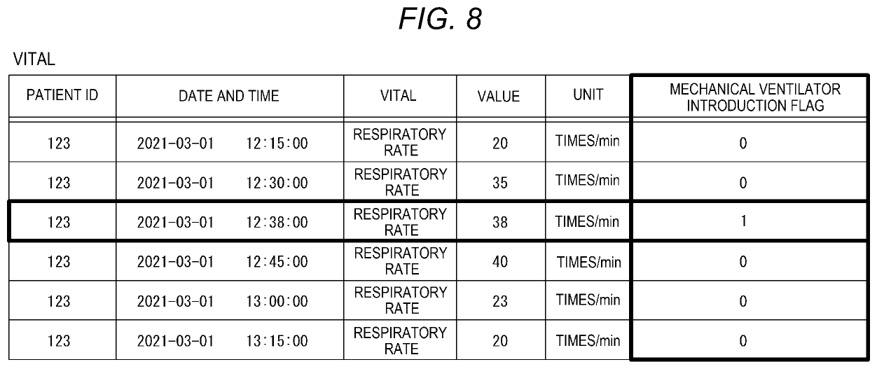

Next, the control unit 21 maps the second medical care information with the occurrence time of the event. Herein, data on the respiratory frequency is indicated on the premise that data on the respiratory frequency in the vital data is included also in the second medical care information. As illustrated in FIG. 8, the control unit 21 may add a flag region for mapping data in the vital data with the occurrence time of the event to the vital data of the respiratory frequency stored in the storage unit 22. For example, the control unit 21 assigns a flag indicating that the mechanical ventilator has been introduced to the vital data measured at each time. In FIG. 8, the data in the line in which the field of “mechanical ventilator introduction flag” is “1” indicates data at the timing when the introduction of the mechanical ventilator is estimated. In a case where vital data at the timing when the introduction of the mechanical ventilator is estimated has not been measured, the control unit 21 may add data at the time when the introduction of the mechanical ventilator was estimated by interpolation. Similar to the above, mapping of data included in the second medical care information other than the respiratory frequency with the occurrence time of the event may be performed.

### Example of Event and Medical Care Information Relating to Event

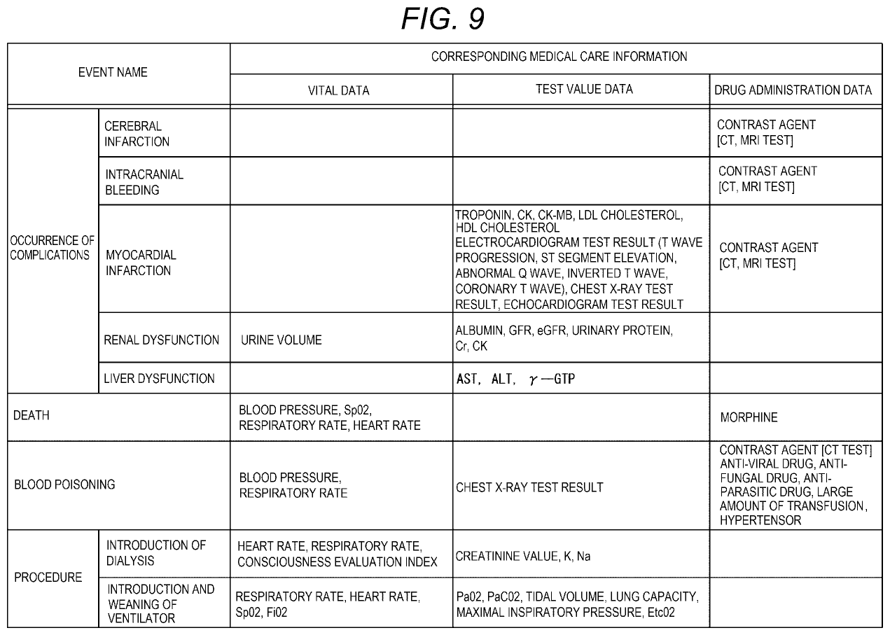

FIG. 9 is a diagram illustrating events and data items in first medical care information relating to each event. Hereinafter, first medical care information relating to several events will be described. The control unit 21 may estimate, with respect to an event, time when the event has occurred by only using one or a plurality of specific data items of first medical care information corresponding to the event.

For a definitive diagnosis of cerebral infarction and intracranial bleeding, photographing such as CT of the head is generally performed. For example, in the CT test of the head, the brain is photographed while injecting a contrast agent, so that the flow of blood in the brain can be observed. Accordingly, as for the events of the occurrence of cerebral infarction and the occurrence of intracranial bleeding, it is possible to estimate the occurrence time of the event by using data at the time when the contrast agent has been administered included in the drug administration data.

Moreover, for example, in a diagnosis of myocardial infarction, the electrocardiography, the blood test, and the imaging test are performed. In a case of myocardial infarction, a waveform specific to the myocardial infarction is generated in the electrocardiogram. Moreover, at the onset of myocardial infarction, rises in various enzymes such as troponin T, CK-MB, and the like in the blood appear. In addition, in the chest X-RAY test, an image of congestion in the lung, cardiac enlargement, or the like is photographed. Accordingly, in a case where the data of these test values are included as data items of the first medical care information, it is assumed that the myocardial infarction has occurred before the tests are performed.

Moreover, in a case where the event is death, it is possible to estimate the time of death from the vital data of the blood pressure, oxygen saturation (SpO₂), the respiratory rate, the heart rate, and the like.

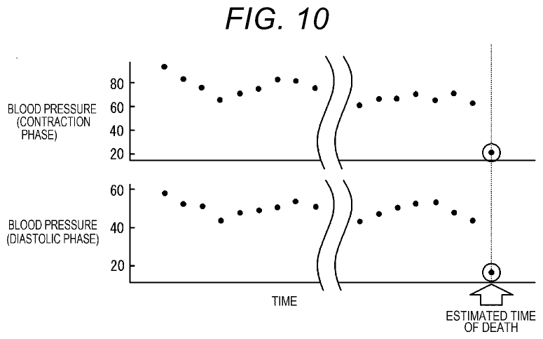

As one example, the control unit 21 can estimate the time when the patient died from data on the blood pressure alone. FIG. 10 illustrates one example of time-series data of the blood pressure in a contraction phase and a diastolic phase. It is possible to estimate that the time when the blood pressure value decreases to nearly 0 is the time when the patient died. Similarly, the control unit 21 can estimate the time of death from the respiratory rate and the heart rate alone.

If the time of death cannot be determined from the drop in the blood pressure value alone, unlike the above, the control unit 21 may estimate the time when the event has occurred from the combination with other vital data such as the respiratory rate or the heart rate.

In a case where the event is blood poisoning, it is possible to determine that the occurrence of blood poisoning is highly probable if two requirements among three requirements that a consciousness disorder is present, the blood pressure in the contraction phase is equal to or less than 100 mmHg, and the respiratory rate for one minute is 22 times or more are satisfied. In addition, for a definitive diagnosis of blood poisoning, imaging diagnoses such as the chest X-RAY test and the CT test are performed. Accordingly, it is possible to set the blood pressure and the respiratory rate included in the vital data, the chest X-RAY test result included in the test value data, and the contrast agent for CT test included in the drug administration data, as data items of the first medical care information relating to the blood poisoning.

### Estimation of Occurrence Time of Event Using Machine Learning

As for a specific event, in a case where more than a given number of precise or highly accurate information on the occurrence time of the event and medical care information are present (i.e., where both more than the given number of precise or highly accurate information on the occurrence time of the event and the medical care information are present), machine learning can also be used in the course of the estimation of occurrence time of the event. The precise event occurrence time has been input to the electronic medical record or other medical information system in some cases. Moreover, the introduction time of the ventilator can be predicted with relatively high accuracy to some extent from the timing when the measurement item relating to breathing has been added to the vital information. The relatively high accuracy of the occurrence time of the event indicates that the recorded occurrence time has a temporal resolution higher than the unit of day, more preferably, a temporal resolution higher than the unit of hour.

The medical information processing device 11 or another dedicated device may generate a learned model (first learned model) for event occurrence time estimation by executing machine learning with medical care information related to the occurrence of an event as input information and the occurrence time of the event as an output. The generated learned model can be stored in the storage unit 22. As for an event in which the precise occurrence time is unclear, the control unit 21 inputs data on first medical care information including the data item the same as that of the medical care information at the learning to the learned model for event occurrence time estimation to allow the learned model to estimate the time when the event has occurred.

As the above, the medical information processing device 11 uses machine learning in some cases, similar to the prediction device 13. However, an occurrence of a future event is predicted from medical care information on a patient in the prediction that is performed by the prediction device 13 in FIG. 1, whereas the time of the occurrence about the specific event having already occurred is estimated in the estimation of the time that is performed by the medical information processing device 11 in the mapping process, which is different from the prediction that is performed by the prediction device 13 in FIG. 1.

### Estimation of Occurrence Time of Another Event Based on Estimated Occurrence Time of Event

The control unit 21 can use event information (first event information) on an event (first event) the occurrence time of which has been estimated as first medical care information for estimating occurrence time of another event (second event). For example, the control unit 21 can set the introduction of a mechanical ventilator as a first event, and set death as a second event. In other words, the control unit 21 may estimate the introduction time of the mechanical ventilator, set information including the estimated introduction time of the mechanical ventilator as first medical care information, and estimate time of death of the patient.

As has been described in the foregoing, with the present embodiment, the occurrence time of the event is estimated with a temporal resolution higher than the unit of day based on the first medical care information, and the second medical care information and the estimated occurrence time of the event are mapped with each other. Accordingly, it is possible to generate data for machine learning in which the event occurrence of the patient is mapped with the second medical care information with a temporal resolution higher than the unit of day. Using the data for machine learning allows the learning device 12 to perform the machine learning and generate a learned model. In addition, the prediction device 13 inputs third medical care information that is medical care information on a patient to be predicted to the generated learned model to allow the occurrence of an event in the patient to be predicted with a temporal resolution higher than the unit of day. Example in Which Respective Devices Are Dispersedly Arranged

In the abovementioned embodiment, the medical information processing device 11, the learning device 12, the prediction device 13, the electronic medical record system 14, the vital data management server 15, the drug administration data management server 16, and the test value data management server 17 can be located in the medical institution. However, these devices can be dispersedly arranged in a geographically separated manner.

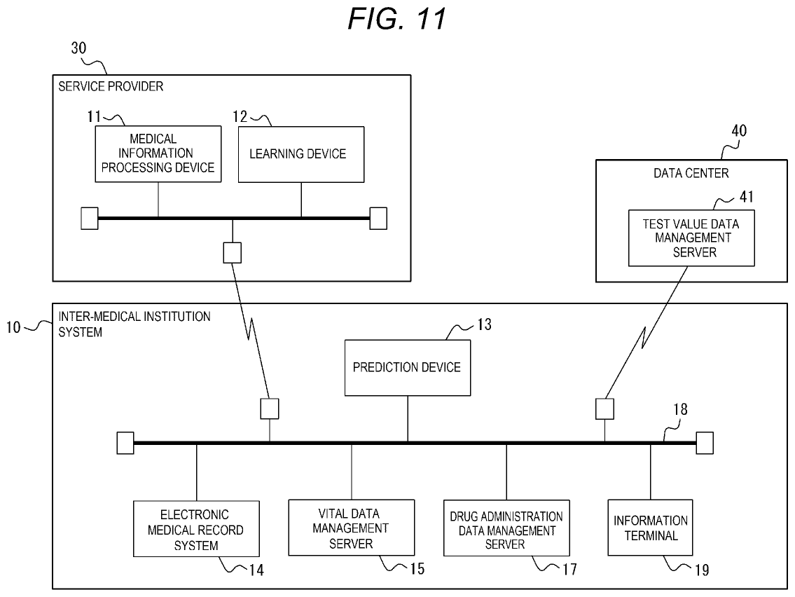

In the example illustrated in FIG. 11, the medical information processing device 11 and the learning device 12 may be located in a base of a service provider 30 that provides the learned model. In order to increase the accuracy of machine learning, a large amount of data for learning including medical care information and event information is preferable. Therefore, the service provider 30 preferably collects information from a plurality of medical institutions in some cases. The service provider 30 may be a provider that provides service or one among the plurality of the medical institutions that employ this system. The system of the service provider 30 may be connected to the plurality of the inter-medical institution systems 10 by a dedicated channel, the Internet, or wide-area communication units such as a virtual private network (VPN).

Moreover, the test value data may be managed by a test value data management server 41 that is disposed in a data center 40 of a provider that provides a test device, not in the server in the inter-medical institution system 10. In this case, the function of the test value data management server 41 may be provided as cloud service. The test value data management server 41 of the provider can manage the test value data of the plurality of the medical institutions. The medical institution can use the test value data management server 41 as if the test value data management server 41 is located in the medical institution. The data center 40 may be connected to the plurality of the inter-medical institution systems 10 by a dedicated channel, the Internet, or a wide-area communication unit such as a virtual private network (VPN).

The arrangement of the devices illustrated in FIG. 11 is one example, and the respective devices can be arranged in various forms. Moreover, the functions of the respective devices, and the medical care information that is managed by the respective devices can be divided or integrated in various forms. The medical institution can manage other medical care data by using the external cloud service, instead of the test value data or in addition to the test value data.

Although the embodiment according to this disclosure has been described based on the respective drawings and the examples, it should be noted that various modifications and changes can be made by persons skilled in the art based on this disclosure. Accordingly, it should be noted that these various modifications and changes are included in the range of this disclosure. For example, the respective constituent units, the functions included in the respective steps, and the like can be rearranged without causing the logical contradictions, and a plurality of constituent units or steps can be combined as one, or can be divided. The embodiment according to this disclosure has been described by focusing on the device, the embodiment according to this disclosure can be implemented as a method including steps that the respective constituent units of the device execute. The embodiment according to this disclosure can be implemented as a method that is executed by a processor provided to the device, a program, or a storage medium in which the program is recorded. It should be understood that these are included in the range of this disclosure. Moreover, the specific event and medical care information having been described in the abovementioned embodiment are merely indicated as examples. This disclosure can be applied to various events and medical care information.

Moreover, in the abovementioned embodiment, information on the date when the event has occurred is set as first temporal information, and time information included in the medical care information and having a temporal resolution higher than the unit of day is set as second temporal information. However, the combination of the first temporal information and the second temporal information in this disclosure are not limited the first temporal information and the second temporal information as disclosed. For example, in a case where occurrence time of an event registered in a medical information system is specified in the unit of hour, and medical care information relating to the event is specified in the unit of minute, it is also possible to set information on the occurrence time of the event in the unit of hour as first temporal information, and the time information in the unit of minute in the medical care information as second temporal information.

The detailed description above describes embodiments of a medical information processing method, a medical information processing device, and a program. These disclosed embodiments represent examples of the medical information processing method, the medical information processing device, and the program disclosed here. The invention is not limited, however, to the precise embodiments and variations described. Various changes, modifications and equivalents can be effected by one skilled in the art without departing from the spirit and scope of the invention as defined in the accompanying claims. It is expressly intended that all such changes, modifications and equivalents which fall within the scope of the claims are embraced by the claims.

What is claimed is:

1. A medical information processing method that is executed by a processor, the medical information processing method comprising:
acquiring event information registered in a medical information system and including a patient identifier (ID), the patient ID including identification information on a patient, identification information on an event, and first temporal information indicating an occurrence time of the event;
acquiring, by using the patient ID and the first temporal information, first medical care information including second temporal information with a temporal resolution higher than that of the first temporal information related to the patient; and
estimating, based on the first medical care information, the occurrence time of the event with the temporal resolution higher than that of the first temporal information.

2. The medical information processing method according to claim 1, wherein the first medical care information includes vital data on the patient.

3. The medical information processing method according to claim 2, further comprising:
estimating the occurrence time of the event based on the second temporal information when a value of data of a predetermined data item in the vital data has achieved a predetermined change.

4. The medical information processing method according to claim 2, further comprising:
estimating the occurrence time of the event based on the second temporal information when the acquired data item in the vital data has been changed.

5. The medical information processing method according to claim 1, wherein the first medical care information includes drug administration data indicating information on administration of a drug performed with respect to the patient.

6. The medical information processing method according to claim 1, wherein the first medical care information includes test value data indicating a result of a test performed with respect to the patient.

7. The medical information processing method according to claim 1, further comprising:
generating the first medical care information including time-series data on a time-series basis by the second temporal information; and
estimating the occurrence time of the event based on a change in a time interval in which the time-series data is generated.

8. The medical information processing method according to claim 1, wherein the first medical care information includes text data input into an electronic medical record or another medical system, and the second temporal information when the text data has been input into the electronic medical record or the another medical system.

9. The medical information processing method according to claim 1, further comprising:
estimating the occurrence time of the event based on a combination of two or more types of the first medical care information.

10. The medical information processing method according to claim 1, further comprising:
mapping the event that includes a specific event with one or more specific data items included in the first medical care information; and
estimating the occurrence time of the specific event based on only the specific data item.

11. The medical information processing method according to claim 1, further comprising:
estimating the occurrence time of the event using a first learned model in which the first medical care information is set as an input and the occurrence time of the event is set as an output.

12. The medical information processing method according to claim 1, further comprising:
setting the event as a first event;
setting information including the first event and the estimated occurrence time as first event information;
using the first event information as the first medical care information; and
estimating occurrence time of a second event different from the first event.

13. The medical information processing method according to claim 1, further comprising:
acquiring second medical care information that is used as input data of a machine learning model that predicts occurrence of the event; and
mapping the second medical care information with the estimated occurrence time of the event.

14. The medical information processing method according to claim 13, further comprising:
mapping the second medical care information with the occurrence time of the event by adding information indicating the occurrence time of the event to the second medical care information.

15. The medical information processing method according to claim 13, further comprising:
setting the event information on a plurality of patients and the second medical care information mapped with the occurrence time of the event as teacher data; and
generating a second learned model in which the second medical care information is set as an input and the event information is set as an output using the teacher data.

16. The medical information processing method according to claim 15, further comprising:
acquiring third medical care information on a specific patient;
inputting the third medical care information into the second learned model; and
predicting an occurrence of an event related to the specific patient.

17. The medical information processing method according to claim 1, wherein the medical information system is an electronic medical record system.

18. The medical information processing method according to claim 1, wherein the first medical care information includes two or more of:
drug administration data indicating information on administration of a drug performed with respect to the patient;
test value data indicating a result of a test performed with respect to the patient; and
text data input into an electronic medical record or another medical system, and the second temporal information when the text data has been input into the electronic medical record or the another medical system.

19. A medical information processing device comprising:
a processor configured to:
acquire event information registered in a medical information system and including a patient identifier (ID), the patient ID including identification information on a patient, identification information on an event, and first temporal information indicating occurrence time of the event;
acquire, by using the patient ID and the first temporal information, first medical care information including second temporal information with a temporal resolution higher than that of the first temporal information related to the patient; and
estimate, based on the first medical care information, the occurrence time of the event with the temporal resolution higher than that of the first temporal information.

20. A non-transitory computer readable program that causes a computer to execute a process comprising:
acquiring event information registered in a medical information system and including a patient identifier (ID), the patient ID including identification information on a patient, identification information on an event, and first temporal information indicating occurrence time of the event;
acquiring, by using the patient ID and the first temporal information, first medical care information including second temporal information with a temporal resolution higher than that of the first temporal information related to the patient; and
estimating, based on the first medical care information, the occurrence time of the event with the temporal resolution higher than that of the first temporal information.
